SEQUENCE LISTING (11 sequences)

SEQ ID NO: 1 - protein, 26 aa, Apis mellifera
GIGAVLKVLTTGLPALISWIKRKRQQ

SEQ ID NO: 2 - DNA, 20 bp, Artificial Sequence
  TNFaF
catccgttctctacccagcc

SEQ ID NO: 3 - DNA, 20 bp, Artificial Sequence
  TNFaR
aattctgagcccggagttgg

SEQ ID NO: 4 - DNA, 20 bp, Artificial Sequence
  NFkBF
ccttgaagggatttccctcc

SEQ ID NO: 5 - DNA, 20 bp, Artificial Sequence
  NFkBR
ggagggaaatcccttcaagg

SEQ ID NO: 6 - DNA, 23 bp, Artificial Sequence
  MCP1F
tgatcccaatgagtaggctggag

SEQ ID NO: 7 - DNA, 23 bp, Artificial Sequence
  MCP1R
atgtctggacccattccttcttg

SEQ ID NO: 8 - DNA, 22 bp, Artificial Sequence
  MMP9F
gccactactgtgcctttgagtc

SEQ ID NO: 9 - DNA, 22 bp, Artificial Sequence
  MMP9R
ccctcagagaatcgccagtact

SEQ ID NO: 10 - DNA, 20 bp, Artificial Sequence
  GAPDHF
agtgccagcctcgtctcata

SEQ ID NO: 11 - DNA, 20 bp, Artificial Sequence
  GAPDHR
ggtaaccaggcgtccgatac

(19) United States
(12) Patent Application Publication
(10) Pub. No.: US 2022/0233457 A1
(43) Pub. Date: Jul. 28, 2022

(54) COMPOSITION FOR PREVENTING, AMELIORATING, OR TREATING CEREBROVASCULAR DISEASE COMPRISING MELITTIN OR MAGNETIC IRON OXIDE NANOPARTICLE LOADED WITH MELITTIN AS EFFECTIVE COMPONENT

(71) Applicant: DAEGU CATHOLIC UNIVERSITY INDUSTRY ACADEMIC COOPERATION FOUNDATION, Gyeongsangbuk-do (KR)

(72) Inventors: Sung Won YOUN, Daegu (KR); Huy Duc VU, Daegu (KR)

(21) Appl. No.: 17/512,937

(22) Filed: Oct. 28, 2021

(30) Foreign Application Priority Data
Jan. 27, 2021 (KR) 10-2021-0011342

(51) Int. Cl. A61K 9/51; A61K 38/17; A23L 33/18

(52) U.S. Cl. CPC A61K 9/5115; A23L 33/18; A61K 38/1767

(57) ABSTRACT

A method for treating a cerebrovascular disease according to an embodiment of the present disclosure includes administering a composition comprising at least one of melittin and magnetic iron oxide nanoparticle loaded with melittin as an effective component to a subject in need thereof. A composition according to an embodiment of the present disclosure for preventing, ameliorating, or treating a cerebrovascular disease includes melittin or magnetic iron oxide nanoparticle loaded with melittin as an effective component. The melittin or magnetic iron oxide nanoparticle loaded with melittin not only can reduce the diameter of cerebral arteries, increase the thickness of cerebral arteries, increase the content of elastin and smooth muscles, and reduce the content of abnormal collagen but also has an effect of suppressing the expression of the factors which mediate an inflammatory response.

### CROSS-REFERENCE TO RELATED APPLICATION(S)

This application claims the benefit under 35 USC § 119(a) of Korean Patent Application No. 10-2021-0011342, filed on Jan. 27, 2021, in the Korean Intellectual Property Office, the entire disclosure of which is incorporated herein by reference for all purposes.

### BACKGROUND

### 1. Technical Field

The present invention relates to a composition for preventing, ameliorating, or treating a cerebrovascular disease comprising melittin or magnetic iron oxide nanoparticle loaded with melittin as an effective component.

### 2. Background Art

Cerebral aneurysm is a disease showing an abnormal bulge in a brain blood vessel caused by weakness in the vessel wall. It indicates a condition that the inner elastic lining and media constituting the interior side of the brain blood vessel are damaged and lost, resulting in the ballooning of the blood vessel wall. More than 90% of cerebral aneurysms are found in major cerebral arteries at the base, which are referred to as the circle of Willis. The rest are found in thin distal cerebral arteries (e.g., blood vessels close to the heart are called proximal vessels while blood vessels far from heart are called distal vessels, and the vessels are thinner as they are more distant from the heart and blood is directly supplied to the brain from the distal vessels) or arteries covering the occipital area or medulla oblongata in the brain. The cerebral aneurysm is less than 10 mm in size in most cases. However, larger sized aneurysm may occur and aneurysms larger than 25 mm are called “giant aneurysm.” Depending on the shape, aneurysms are classified into the saccular aneurysm, fusiform aneurysm, and dissecting aneurysm.

The exact causes of cerebral aneurysms remain unknown. However, as it is mainly found in arterial branches and proximal segments, it is believed that, in an area experiencing high pressure according to hemodynamics, acquired cracks develop in the blood vessel wall to cause an occurrence and growth of an aneurysm. The cerebral aneurysm usually occurs in the population aged between the 40s and 60s, and multiple aneurysms can be found in about 20% of the patients with cerebral aneurysms. Although rare, aneurysm also occurs when there is inflammation of blood vessels, damaged blood vessel wall due to trauma, genetic problems in the blood vessel wall, or the like. Cerebral arteriovenous malformation or cerebrovascular diseases like Moyamoya disease may also accompany the aneurysm. Although there are reports indicating that a cerebral aneurysm is caused by smoking, high blood pressure, or drug abuse, a clear cause of the cerebral aneurysm is yet to be found.

Diagnostic evaluation of the cerebral aneurysm can be made by brain computed tomography (CT), brain nuclear resonance imaging (MRI), or catheter cerebral angiography. Thanks to the recent progress in the technology, diagnosis and therapeutic planning for cerebral aneurysm can be made based on the non-invasive evaluation such as brain CT or brain MRI. However, catheter cerebral angiography, the invasive procedure is still the gold standard for diagnosis and therapeutic planning. In the case of endovascular coil embolization, catheter cerebral angiography is established as a monitoring tool for the endovascular therapy that is more frequently employed than surgical aneurysm clipping. Based on the catheter angiographic findings before, during, and after endovascular coil embolization, the cerebral aneurysm can be diagnosed, monitored, and confirmed to be safely occluded. In a rare case of aneurysm rupture and bleeding, the brain CT or MRI can reveal the intracranial bleeding, but the aneurysmal sac itself may not be seen as it is compressed by hematoma, and thus the definite diagnosis is made by carrying out the examination again after 2 weeks or so. Acute intracranial bleedings such as subarachnoid hemorrhage, intracerebral hemorrhage, intraventricular hemorrhage, and its related complications such as vasospasm, and hydrocephalus can be diagnosed by brain imaging modalities. Although subarachnoid hemorrhage is sometimes not detected under imaging modalities, when the ruptured cerebral aneurysm is highly suspected based on other symptoms, small subarachnoid hemorrhage can be diagnosed by cerebrospinal fluid tapping.

Melittin is the main component of honeybee venom, i.e., up to 40 to 50% of the venom, and it consists of 26 amino acids. It has been reported that melittin has various effects like suppressing the growth of bacteria, necrosis, anti-inflammation, pain relief, enhancing immunity, or the like. For example, in Korean Patent Application Publication No. 2019-0127609, “Targeting of M2-like tumor-associated macrophages with melittin-based pro-apoptotic peptide” is described, and, in Korean Patent Registration No. 2042059, “Composition for prevention, treatment, and amelioration of cancer comprising melittin nanoparticle” is disclosed. However, the composition of the present invention for preventing, ameliorating, or treating a cerebrovascular disease comprising melittin or magnetic iron oxide nanoparticle loaded with melittin as the effective component is not described before.

### SUMMARY

The present invention is devised under the circumstances that are described above, and the present invention provides a composition for preventing, ameliorating, or treating a cerebrovascular disease comprising melittin or magnetic iron oxide nanoparticle loaded with melittin as an effective component. As it is found in the present invention that melittin or magnetic iron oxide nanoparticle loaded with melittin of the present invention not only can reduce the diameter of cerebral arteries, increase the thickness of cerebral arteries, increase the content of elastin and smooth muscles, and reduce the content of abnormal collagen but also has an effect of suppressing the expression of MMP-9, MCP-1, CD68, TNF-α, and NFκB which mediate an inflammatory response, the present invention is completed accordingly.

To achieve the object described above, the present invention provides a composition for preventing or treating a cerebrovascular disease comprising melittin or magnetic iron oxide nanoparticle loaded with melittin as an effective component.

The present invention also provides a functional health food composition for preventing or ameliorating cerebrovascular disease comprising melittin or magnetic iron oxide nanoparticle loaded with melittin as an effective component.

The present invention relates to a composition for preventing, ameliorating, or treating a cerebrovascular disease comprising melittin or magnetic iron oxide nanoparticle loaded with melittin as an effective component. The melittin or magnetic iron oxide nanoparticle loaded with melittin of the present invention not only can reduce the diameter of cerebral arteries, increase the thickness of cerebral arteries, increase the content of elastin and smooth muscles, and reduce the content of abnormal collagen but also has an effect of suppressing the expression of MMP-9, MCP-1, CD68, TNF-α, and NFκB which mediate an inflammatory response.

### BRIEF DESCRIPTION OF THE DRAWINGS

A of FIG. 1 illustrates the structure of an iron oxide nanoparticle loaded with melittin (MeLioN), which has been formed by linking iron oxide nanoparticle, L-arginine, and melittin. B of FIG. 1 shows the photographic image of an iron oxide nanoparticle (ION), an L-arginine-linked nanoparticle (LION), and a nanoparticle loaded with melittin (MeLioN), in which the image was obtained by transmission electron microscope (EM) or scanning electron microscope (SEM).

FIG. 2 illustrates the process of inducing cerebral aneurysm, in which, under a high salt diet, unilateral nephrectomy was carried out at week 1; stereotaxic elastase injection into a basal cistern and continuous subcutaneous infusion of angiotensin II were carried out at week 2; and, after week 2, iron oxide nanoparticle (ION), melittin (MEL), or magnetic nanoparticle loaded with melittin (MeLioN) was administered in each group 5 times per 3 days, with a 2.5 mg/kg/dose, and then the brain tissues were harvested at week 4.

FIGS. 3A to 3C illustrate the mouse brain arterial wall administered with melittin or magnetic iron oxide nanoparticle loaded with melittin, in which determination was made by H&E staining to examine the morphology of cerebral arteries. Specifically, FIG. 3A represents the photographic image of the H&E staining, FIG. 3B represents the result of determining the diameter of cerebral arteries, and FIG. 3C represents the result of determining the thickness of cerebral arteries. H denotes a healthy group with a regular diet, D denotes a diseased group induced to have a cerebral aneurysm for which high salt diet-unilateral nephrectomy-cisternal elastase infusion have been carried out, ION denotes a group induced to have a cerebral aneurysm and simultaneously administered with iron oxide nanoparticle only, MEL denotes a group induced to have a cerebral aneurysm and simultaneously administered with melittin only, and MeLioN denotes a group induced to have a cerebral aneurysm and simultaneously administered with magnetic iron oxide nanoparticle loaded with melittin.

In the diseased group induced to have a cerebral aneurysm to which high salt diet-unilateral nephrectomy-cisternal elastase infusion have been carried out, the diameter of cerebral arteries showed a significant increase and the thickness of cerebral arteries showed a significant decrease in comparison to the healthy group with a regular diet with a p-value less than 0.001 (###). *** indicates that compared to the cerebral aneurysm group (D), the diameter of cerebral arteries shows a statistically significant decrease in the MeLioN group administered with magnetic iron oxide nanoparticle loaded with melittin, and the thickness of cerebral arteries shows an increase in the group administered with iron oxide nanoparticle only (ION), the group administered with melittin only (MEL), and also the group administered with magnetic iron oxide nanoparticle loaded with melittin (MeLioN) with p<0.001. ns denotes a change that is not significantly different.

FIG. 4 shows the mouse brain arterial walls after the administration of magnetic iron oxide nanoparticle loaded with melittin, in which evaluation was based on the fractional ratio of elastin by Verhoff Van Gieson staining of mouse brain arterial wall. H denotes a healthy group with a regular diet, D denotes a diseased group induced to have a cerebral aneurysm for which a high salt diet—unilateral nephrectomy—cisternal elastase infusion have been carried out, ION denotes a group induced to have a cerebral aneurysm and simultaneously administered with iron oxide nanoparticle only, MEL denotes a group induced to have a cerebral aneurysm and simultaneously administered with melittin only, and MeLioN denotes a group induced to have a cerebral aneurysm and simultaneously administered with magnetic iron oxide nanoparticle loaded with melittin. *** indicates that, in comparison to the healthy group (H), the fractional ratio of elastin becomes significantly lower in the diseased group induced to have a cerebral aneurysm (D), the group administered with iron oxide nanoparticle only (ION), and also the group administered with melittin only (MEL) with p-value less than 0.001. However, the elastin content of the healthy group (H) was not statistically different from that of the MeLioN-treated group. ns means a change that is not statistically significant.

FIG. 5 shows mouse brain arterial wall after the administration of magnetic iron oxide nanoparticle loaded with melittin (MeLioN), in which determination was made by using an animal model, based on examination of fractional ratio of collagen by Trichrome staining of the tissues. H denotes a healthy group with a regular diet, D denotes a diseased group induced to have a cerebral aneurysm for which a high salt diet-unilateral nephrectomy-cisternal elastase infusion have been carried out, ION denotes a group induced to have a cerebral aneurysm and simultaneously administered with iron oxide nanoparticle only, MEL denotes a group induced to have a cerebral aneurysm and simultaneously administered with melittin only, and MeLioN denotes a group induced to have a cerebral aneurysm and simultaneously administered with magnetic iron oxide nanoparticle loaded with melittin. The fractional ratio of collagen becomes significantly higher in the diseased group (D) in comparison to the healthy group (H) with a p-value less than 0.001 (###). The fractional ratio of collagen becomes significantly lower in the group administered with magnetic iron oxide nanoparticle loaded with melittin (MeLioN) in comparison to the diseased group (D) with a p-value less than 0.001(***). However, the collagen content of the diseased group was not statistically different from that of the ION-treated or MEL-treated group, respectively. ns means a change that is not statistically significant.

FIG. 6 shows the result of determining the fractional ratio of smooth muscle cells after the administration of magnetic iron oxide nanoparticle loaded with melittin, in which determination was made, by using an animal model, based on staining of cerebrovascular tissues. H denotes the healthy group with a regular diet, D denotes the diseased group induced to have a cerebral aneurysm for which high salt diet—unilateral nephrectomy—cisternal elastase infusion have been carried out, ION denotes the group induced to have a cerebral aneurysm and simultaneously administered with iron oxide nanoparticle only, MEL denotes the group induced to have a cerebral aneurysm and simultaneously administered with melittin only, and MeLioN denotes the group induced to have a cerebral aneurysm and simultaneously administered with magnetic iron oxide nanoparticle loaded with melittin.

FIGS. 7A to 7D show the suppressed expression level of MMP-9 in brain arterial wall according to the administration of melittin (MEL) or magnetic iron oxide nanoparticle loaded with melittin (MeLioN) of the present invention, in which FIG. 7A is the result of immunohistochemical staining, FIG. 7B is the statistical graph of the result of FIG. 7A, FIG. 7C is the result of real-time polymerase chain reaction (PCR), and FIG. 7D is the result of Western blot analysis. H denotes the healthy group with a regular diet, D denotes the diseased group induced to have a cerebral aneurysm for which high salt diet—unilateral nephrectomy—cisternal elastase infusion have been carried out, ION denotes the group induced to have a cerebral aneurysm and simultaneously administered with iron oxide nanoparticle only, MEL denotes the group induced to have a cerebral aneurysm and simultaneously administered with melittin only, and MeLioN denotes the group induced to have a cerebral aneurysm and simultaneously administered with magnetic iron oxide nanoparticle loaded with melittin. In the figure, 0, level 0 (distribution of the expression was not found); 1, level 1 (distribution of the expression was found in less than half of the arterial wall circumference); 2, level 2 (distribution of the expression was found in more than half of the arterial circumference). # indicates that in comparison to the healthy group (H), the expression level of MMP-9 is significantly higher in the diseased group (D) induced to have a cerebral aneurysm with p<0.05. * indicates that in comparison to the diseased group (D) induced to have a cerebral aneurysm, the expression level of MMP-9 is significantly lower in the MeLioN group administered with magnetic iron oxide nanoparticle loaded with melittin with p<0.05.

FIGS. 8A to 8F show suppressed expression level of MCP-1 and macrophage (CD68) in brain arterial wall according to the administration of melittin (MEL) or magnetic iron oxide nanoparticle loaded with melittin (MeLioN) of the present invention, in which FIG. 8A is the result of immunohistochemical staining of MCP-1, FIG. 8C is the statistical graph of the result of FIG. 8A, FIG. 8B is the result of immunohistochemical staining of macrophage (CD68), FIG. 8D is the statistical graph of the result of FIG. 8B, FIG. 8E is the result of real-time PCR of the MCP-1 gene, and FIG. 8F is the result of Western blot analysis of MCP-1 protein. H denotes the healthy group with a regular diet, D denotes the diseased group induced to have a cerebral aneurysm for which high salt diet—unilateral nephrectomy—cisternal elastase infusion have been carried out, ION denotes the group induced to have a cerebral aneurysm and simultaneously administered with iron oxide nanoparticle only, MEL denotes the group induced to have a cerebral aneurysm and simultaneously administered with melittin only, and MeLioN denotes the group induced to have a cerebral aneurysm and simultaneously administered with magnetic iron oxide nanoparticle loaded with melittin. In the figure, 0, level 0 (distribution of the expression was not found); 1, level 1 (distribution of the expression was found in less than half of the arterial wall circumference); 2, level 2 (distribution of the expression was found in more than half of the arterial wall circumference). # indicates that the expression level of MCP-1 is significantly higher in the diseased group (D) induced to have a cerebral aneurysm in comparison with the healthy group (H) with p<0.05. * indicates that the expression level of MCP-1 is significantly lower in the MeLioN group administered with magnetic iron oxide nanoparticle loaded with melittin in comparison with the diseased group (D) induced to have a cerebral aneurysm with p<0.05.

FIGS. 9A to 9E show suppressed expression level of TNF-α in brain arterial wall according to the administration of melittin (MEL) or magnetic iron oxide nanoparticle loaded with melittin (MeLioN) of the present invention, in which FIG. 9A is the result of immunohistochemical staining of TNF-α, FIG. 9B is the statistical graph of the result of FIG. 9A, FIGS. 9C and 9D are the result of real-time polymerase chain reaction (PCR) of TNF-α gene and NFκB gene, respectively, and FIG. 9E is the result of Western blot analysis TNF-α protein and NFκB protein. H denotes a healthy group with a regular diet, D denotes a group induced to have a cerebral aneurysm for which high salt diet-unilateral nephrectomy-cisternal elastase infusion have been carried out, ION denotes a group induced to have a cerebral aneurysm and simultaneously administered with iron oxide nanoparticle only, MEL denotes a group induced to have a cerebral aneurysm and simultaneously administered with melittin only, and MeLioN denotes a group induced to have a cerebral aneurysm and simultaneously administered with magnetic iron oxide nanoparticle loaded with melittin. In the figure, 0, level 0 (distribution of the expression was not found); 1, level 1 (distribution of the expression was found in less than half of the arterial wall circumference); 2, level 2 (distribution of the expression was found in more than half of the arterial wall circumference). # indicates that the expression level of each TNF-α and NFκB is significantly higher in the diseased group (D) induced to have a cerebral aneurysm in comparison to the healthy group (H) with p<0.05. * indicates that the expression level of each TNF-α and NFκB is significantly lower in the MeLioN group administered with magnetic iron oxide nanoparticle loaded with melittin in comparison to the diseased group (D) induced to have a cerebral aneurysm with p<0.05.

FIGS. 10A to 10C show cytotoxicity of melittin-loaded L-arginine-coated iron oxide nanoparticle (MeLioN) and free melittin (MEL) using MTT assay and Hemolysis test. The RAW 264.7 cells were treated with 0.1, 0.5, 1, 2, 4 μg/ml of MeLioN and free melittin for 12 h (FIG. 10A) or 24 h (FIG. 10B). In the 12 h of free melittin treatment, RAW 264.7 cell viability was significantly decreased at 1.0 μg/mL, 2.0 μg/mL and 4.0 μg/mL melittin concentrations. In the 24 h of free melittin treatment, the cell viability was significantly decreased at 0.5 μg/mL, 1.0 μg/mL, 2.0 μg/mL and 4.0 μg/mL melittin concentrations. No significant viability changes were detected at melittin concentration below 1.0 μg/mL (for 12 h) and 0.5 μg/mL (for 24 h). In contrast, the 12 h and 24 h of MeLioN treatment did not decrease RAW 264.7 cell viability until full dose escalation to 4 μg/mL of melittin concentration. The cell viability (%) was expressed as mean±standard error of the mean (n=6) and compared the free melittin and MeLioN-treated groups. ****, p<0.001; ***, p<0.001; **, p<0.01 and *, p<0.05. (FIG. 10C) The mouse blood was treated with 1, 5, 10, 20, 50 μg/mL of MeLioN and free melittin. Hemolysis (%) was expressed as mean±standard error of the mean (n=3) and compared the free melittin and MeLioN-treated groups. MeLioN showed no hemolytic activity at up to 20 μg/mL (1-2%) and approximately 10% at 50 μg/mL, while free melittin showed 100% hemolytic activity at from 1 μg/mL.

### DETAILED DESCRIPTION

The present invention relates to a pharmaceutical composition for preventing or treating a cerebrovascular disease comprising melittin or magnetic iron oxide nanoparticle loaded with melittin as an effective component.

The cerebrovascular disease is preferably anyone selected from a cerebral aneurysm; any intracranial hemorrhage such as subarachnoid hemorrhage, parenchymal hemorrhage and extra-axial hemorrhage; and cerebral infarction caused by cerebral aneurysm, but it is not limited thereto.

The melittin preferably consists of the amino acid sequence of SEQ ID NO: 1, but it is not limited thereto. Even when one or more amino acid residues of the amino acid sequence of SEQ ID NO: 1 are substituted, deleted, or inserted, those derived from melittin composed of the amino acid sequence of SEQ ID NO: 1 and useful for preventing or treating cerebrovascular disease can be freely used without any limitation.

The magnetic iron oxide nanoparticle loaded with melittin is preferably those produced by the method including the following steps:
(1) adding ferrous chloride and ferric chloride, admixed at 1:1 to 1:3 mole ratio, to a solution of L-arginine, and slowly adding over 1 hour a solution of ammonium hydroxide under stirring at 800 to 1200 rpm, 70 to 90° C. to produce a precipitated paramagnetic iron oxide nanoparticle; and
(2) having melittin linked to a surface of the paramagnetic iron oxide nanoparticle which has been produced in the above step (1),
   but it is not limited thereto.

The melittin or magnetic iron oxide nanoparticle loaded with melittin may suppress the expression of at least one gene or protein selected from MMP-9, MCP-1, CD68, TNF-α, and NFκB which mediate an inflammatory response.

The composition of the present invention may further comprise, other than the above effective component, a pharmaceutically acceptable carrier, vehicle, or diluent, and can be prepared in various formulations including an oral formulation and a parenteral formulation. In case of producing a formulation, production is made by using a diluent or a vehicle such as filler, bulking agent, binding agent, moisturizing agent, disintegrating agent, or surfactant that are commonly used for producing a formulation. As for the solid formulation for oral administration, a capsule, a powder, a granule, a tablet, a pill or the like are included, and such solid formulation is produced by mixing at least one compound with one or more vehicles such as starch, calcium carbonate, sucrose, lactose, or gelatin. Furthermore, other than simple vehicles, a lubricating agent such as magnesium stearate or talc can be also used. As for the liquid formulation for oral administration, a suspension, an emulsion, a syrup formulation, an aerosol, or the like can be mentioned. Other than water or liquid paraffin as a commonly used simple diluent, various kinds of a vehicle such as moisturizing agent, sweetening agent, aromatic agent, or preservatives may be included. Examples of a formulation for parenteral administration include a sterilized aqueous solution, anon-aqueous formulation, a suspension, an emulsion, a freeze-dried formulation, and a suppository. As a water-insoluble solvent or a suspending agent, propylene glycol, polyethylene glycol, or vegetable oil such as olive oil, and injectable ester such as ethyl oleate can be used. As a base for a suppository, witepsol, macrogol, tween 61, cacao fat, laurin fat, glycerol, gelatin, or the like can be used. In case of parenteral administration, it is preferable to choose external application on skin, intraperitoneal, rectal, intravenous, muscular, subcutaneous, endometrium injection, or intracerebroventricular injection.

The pharmaceutical composition of the present invention is administered in a pharmaceutically effective amount. As described herein, the expression “pharmaceutically effective amount” means an amount sufficient for treating a disorder at a reasonable benefit-risk ratio that can be applied for medical treatment. The effective dose level may be determined based on a type or severeness of a disorder of a patient, activity of a pharmaceutical, sensitivity to a pharmaceutical, administration period, administration route, excretion ratio, time period for therapy, elements including a pharmaceutical used in combination, and other elements that are well known in the medical field. The composition of the present invention can be administered as a separate therapeutic agent, or it can be administered in combination with other therapeutic agents. It can be administered in order or simultaneously with a conventional therapeutic agent. It can be also administered as single-dose or multi-dose. It is important to administer an amount that allows obtainment of the maximum effect with minimum dose while considering all of the aforementioned elements without having any side effect, and the dosage can be easily determined by a person skilled in the pertinent art.

The dosage of the composition of the present invention may vary depending on body weight, age, sex, health state, diet of a patient, administration period, administration method, excretion rate, and severeness of disorder. The composition of the present invention may be also used either singly or in combination with surgery, radiation therapy, hormone therapy, chemotherapy, or method of using biological response modifiers, or the like.

The present invention further relates to a functional health food composition for preventing or ameliorating cerebrovascular disease comprising melittin or magnetic iron oxide nanoparticle loaded with melittin as an effective component.

The composition is preferably prepared in any one formulation selected from of a powder, a granule, a pill, a tablet, a capsule, a candy, a syrup, or a drink, but it is not limited thereto.

When the functional health food composition is used as a food additive, the effective component can be either directly added or used with other food or food components, and it can be suitably used according to a common method. The mixing amount of effective component can be suitably determined depending on a desired use thereof (i.e., prevention, health promotion, or therapeutic treatment). In general, for producing a food product or a drink, the composition of the present invention is added in an amount of 15 parts by weight or less, and preferably 10 parts by weight of less relative to the raw materials. However, when it is used for a long period of time, e.g., for maintaining health or hygiene, or keeping a good health state or the like, the mixing amount can be less than the aforementioned range, and, as there is no problem in terms of safety, the effective component can be also used in an amount that is more than the aforementioned range.

Type of the food product is not particularly limited. As for an example of the food products to which the extract or a fraction thereof can be added, it can be anyone selected from meats, sausages, breads, chocolates, candies, snacks, cookies, pizza, ramen, other noodles, gums, dairy products including ice cream, various soups, beverages, tea, drinks, alcohol beverages, and vitamin complexes, and it includes any health food products in general sense.

When the composition of the present invention is consumed as a health drink, various flavors or natural carbohydrates may be further included as an additional component like common drinks. Examples of the natural carbohydrates include monosaccharides such as glucose or fructose, disaccharides such as maltose or sucrose, polysaccharides such as dextrin or cyclodextrin, and sugar alcohols such as xylitol, sorbitol, or erythritol. As a sweetening agent, a natural sweetening agent such as thaumatin or stevia extract and a synthetic sweetening agent such as saccharine or aspartame can be used. The ratio of the natural carbohydrates is generally about 0.01 to 0.04 g, and preferably about 0.02 to 0.03 g per 100 g of the composition of the present invention. Other than those described in the above, the composition of the present invention may further comprise various nutritional supplements, a vitamin, an electrolyte, a flavor, a coloring agent, pectinic acid and a salt thereof, alginic acid and a salt thereof, an organic acid, protective colloidal thickening agent, a pH adjusting agent, a stabilizer, a preservative, glycerin, alcohol, and a carbonating agent used for carbonated drink. Other than those, fruit flesh for producing natural fruit juice, fruit juice drink, or vegetable drink can be also comprised. Those components may be used either independently or in combination thereof. The ratio of those additives is generally selected, although it is not critical, from a range of 0.01 to 0.1 part by weight per 100 parts by weight of the composition of the present invention.

Hereinbelow, the present invention is explained in greater detail in view of the Examples. However, the following Examples are given only for a specific explanation of the present invention and it would be evident to a person who has common knowledge in the pertinent art that the scope of the present invention is not limited by them.

### EXAMPLES

### Example 1. Synthesis of Iron Oxide Nanoparticle Loaded with Melittin

(1) Separation and Purification of Melittin from Honeybee Venom
The melittin used in the present invention consists of the following amino acid sequence.

### [SEQ ID NO: 1]

COOH-Gly-Ile-Gly-Ala-Val-Leu-Lys-Val-Leu-Thr-Thr-Gly-Leu-Pro-Ala-Leu-Ile-Ser-Trp-Ile-Lys-Arg-Lys-Arg-Gln-Gln-NH₂

One gram of dried honeybee venom was dissolved in 100 ml distilled water, and filtered using a 0.45 μm syringe filter (Sartorius, USA). After that, by continuously passing through ultra-filtration membranes (30 kDa and 10 kDa), phospholipase A2 was removed and melittin was separated and purified from the honeybee venom.

### (2) Preparation of Melittin Solution

Melittin powder (5 mg) was added at 25° C. to 1.0 ml PBS (pH 7.4) to prepare a melittin solution (5.0 mg/ml). With this 5.0 mg/ml melittin solution, 0.125 mg/ml melittin solution was prepared and used for the group treated with melittin only.

Moreover, for loading onto a surface of iron oxide nanoparticle, 0.36 mM (=1.0 mg/ml) melittin solution was prepared, and this solution was found to be stably maintained at 4° C. for 8 weeks.

(3) Synthesis of L-Arginine-Coated Magnetic Nanoparticle Loaded with Melittin

Ferrous chloride (FeCl₂.4H₂O) and ferric chloride (FeCl₃.6H₂O) were admixed at mole ratio of 1:2, and dissolved in 20 ml of ultrapure L-arginine solution (0.07% (w/v)) which has been deaerated in advance. After that, under vigorous stirring at 1000 rpm, 80° C., 7 ml of 25% (w/v) NH₄OH were slowly added thereto over 1 hour to have complete precipitation. After the reaction, with use of a magnet, arginine-coated nanoparticles were washed 3 times with water and ethanol, and dried in an oven at 60° C. for 24 hours.

On the surface of the obtained paramagnetic iron oxide nanoparticle, melittin (2.5 mg/kg) was loaded. Melittin solution (0.36 mM, 0.5 ml) and suspension of the magnetic nanoparticle (2.5 mg/ml, 0.5 ml) were added to a 4 ml centrifuge tube. The mixture was then continuously stirred for 48 hours at 4° C. to have melittin loaded on the magnetic nanoparticle.

Upon the completion of the loading of melittin, the sample was allowed to stand for 10 minutes followed by centrifuge (150 g, 10 minutes) to separate the supernatant from pellets (i.e., precipitates). Unbound melittin present in the supernatant was carefully removed, and then stored in another container to measure the loading efficiency. After that, the soft pellets were washed 3 times, and the magnetic iron oxide nanoparticle loaded with melittin was dispersed again in 1 mL PBS (pH 7.4) (FIG. 1).

### Example 2. Preparation of Animal Model Induced to have a Cerebral Aneurysm

To determine the effect of the magnetic iron oxide nanoparticle loaded with melittin of the present invention, an animal model was induced to have a cerebral aneurysm. A male C57BL/6J mouse was purchased from Samtako Bio Korea, and, after acclimation for about 1 week, the healthy animals were housed in a polycarbonate cage with random allocation to each test group. Environmental condition for breeding includes a temperature of 22±3° C., relative humidity of 50±10%, lighting hour of 12 hours, and illuminance of 150 to 300 lux. The animals were allowed free access to food and water. Hypertension was induced by a high salt diet, unilateral nephrectomy, and subcutaneous pump infusion of angiotensin II.

Specifically, a male C57BL/6J mouse was anesthetized with isoflurane gas and the left kidney was removed by surgery. One week after the surgery, the mouse was fixed onto a nose cone, anesthetized with isoflurane gas by inhalation, and then fixed on a stereostatic system. By using a 10 μl Hamilton syringe, 10 μl of 1.0 U elastase solution was injected to the right cistern to induce cerebral aneurysm. For elastase injection, 1.0 U elastase was injected through a hole on the right skull, under anesthetization with isoflurane gas by inhalation, by pushing the syringe to a space of cerebrospinal fluid in the right bottom, 2.5 mm behind the bregma (parietal point) and 1.0 mm to the right of the center line, and elastase was injected to the depth of 5.0 min.

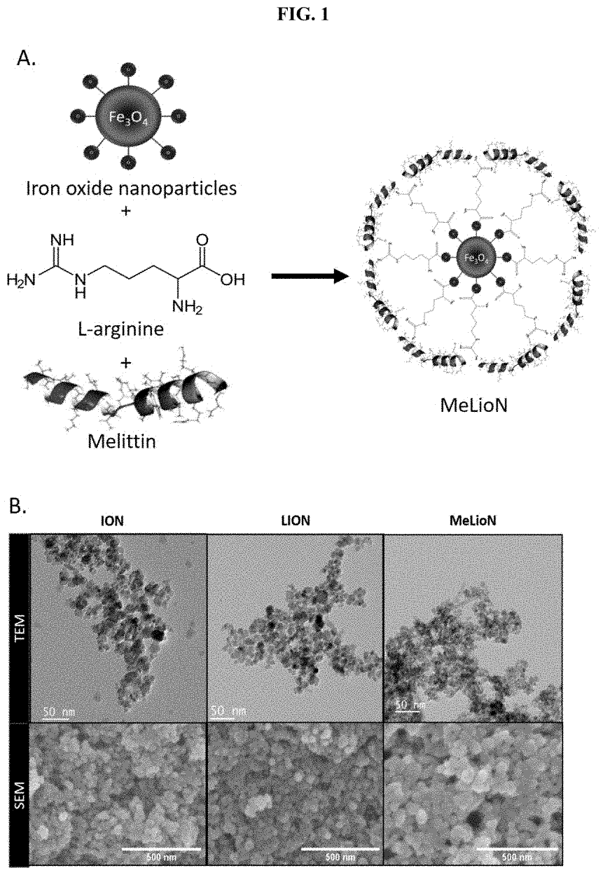

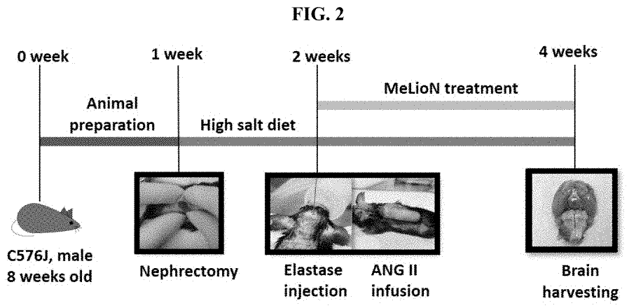

After that, to have a continuous infusion of angiotensin II, an osmotic pump was subcutaneously inserted (1000 ng/kg/min) and cerebral aneurysm was induced by performing a high salt diet (8% NaCl, Enbigo) for 14 days (FIG. 2).

The experimental mice group includes the healthy group (H), diseased group induced to have a cerebral aneurysm (D), group administered with iron oxide nanoparticle (ION), group treated with melittin (MEL), and the group treated with magnetic iron oxide nanoparticle loaded with melittin (MeLioN).

Immediately after the induction of cerebral aneurysm, the group administered with magnetic iron oxide nanoparticle loaded with melittin (MeLioN) was injected, via tail vein, with MeLioN at a dose of 2.5 mg/kg, 5 times every three days. The group administered with melittin (MEL) was injected with 1.0 mg/kg melittin via tail vein, and the group administered with iron oxide nanoparticle (ION) was injected 5 times with suspension of iron oxide nanoparticle (1.25 mg/ml) via tail vein. To the all test groups, the pharmaceutical was administered in an amount of 0.1 ml. The animal brain was harvested at week 4 as shown in FIG. 2, and used as a test sample for the analysis of following Examples 3 to 5.

### Example 3. Analysis of Cerebral Arterial Wall after Administration of Melittin or Magnetic Iron Oxide Nanoparticle Loaded with Melittin of the Present Invention

By using the animal model prepared in above Example 2, hematoxylin and eosin (H&E) staining, Verhoff Van Gieson staining for analyzing the elastin content, Trichrome staining for analyzing the collagen content, and smooth muscle cell staining were carried out to have the histological analysis of the cerebral artery tissues.

The mouse was sacrificed by cardiac perfusion of PBS (4 ml) and 4% paraformaldehyde (PFA, 4 ml). After fixative perfusion, bromophenol blue dye dissolved in 10% gelatin/PBS solution was immediately perfused through the left ventricle. The brain tissues of the mouse were harvested, and then, for an additional histopathological test, the tissues were immersed in 4% PFA for 24 hours in a refrigerator. The mouse brain was dissected into 5 flat pieces based on the location of the cerebral artery of the Willis circle. Mouse brain specimen was then prepared for histopathological visualization and examination.

### (1) Hematoxylin and Eosin (H&E) Staining

According to the visualization of cell nucleus, cytoplasm, and extracellular matrix by H&E staining of cerebral artery tissues, a change in the structure and shape of the cerebral artery was analyzed. To examine a change in the brain arterial wall, an immunohistochemical microscopic image was scanned and the diameter and thickness of the artery were measured by using Case Viewer software.

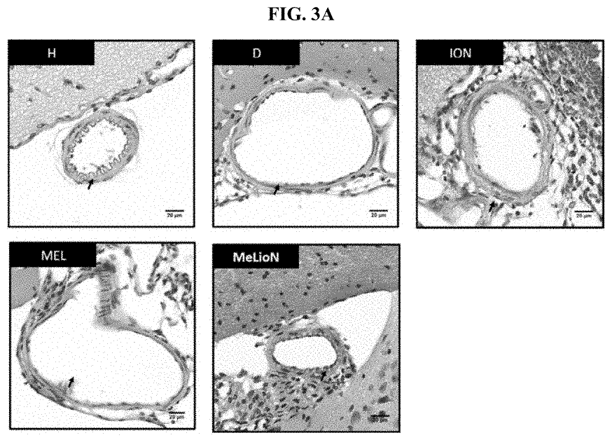

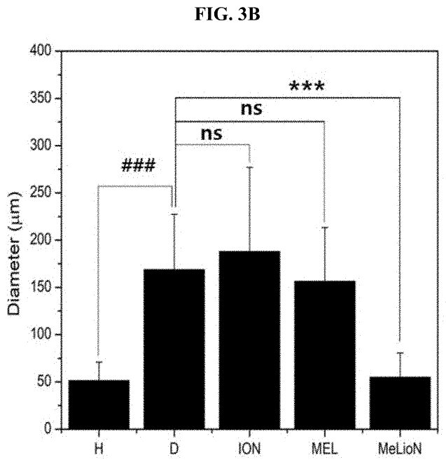

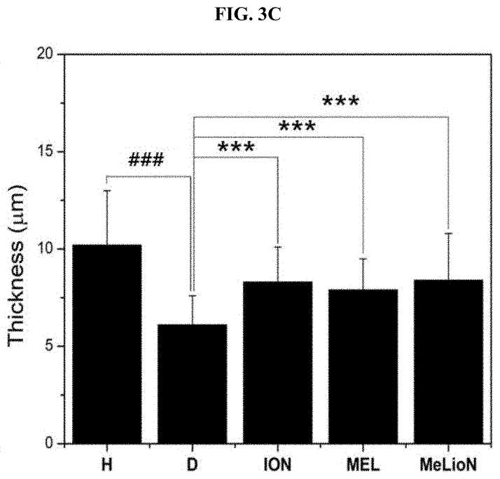

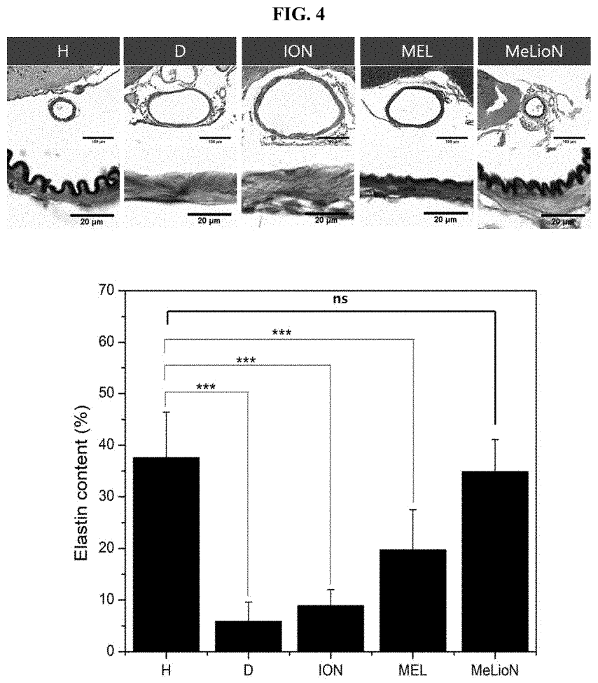

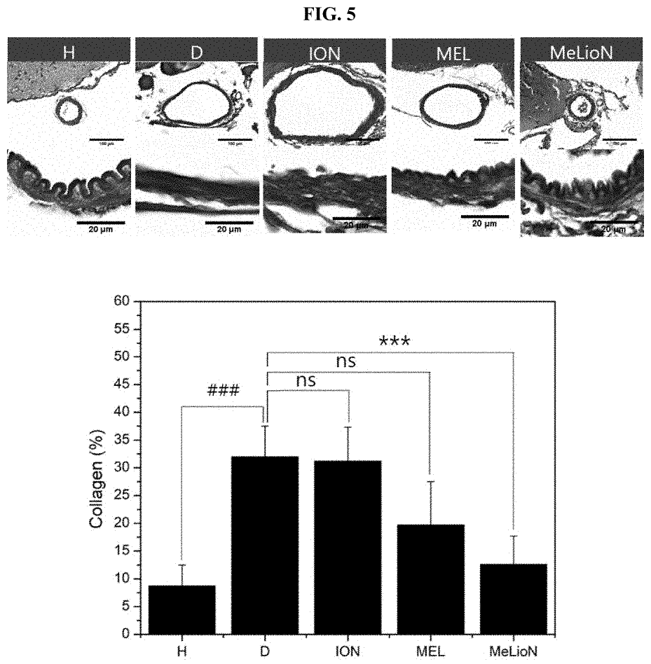

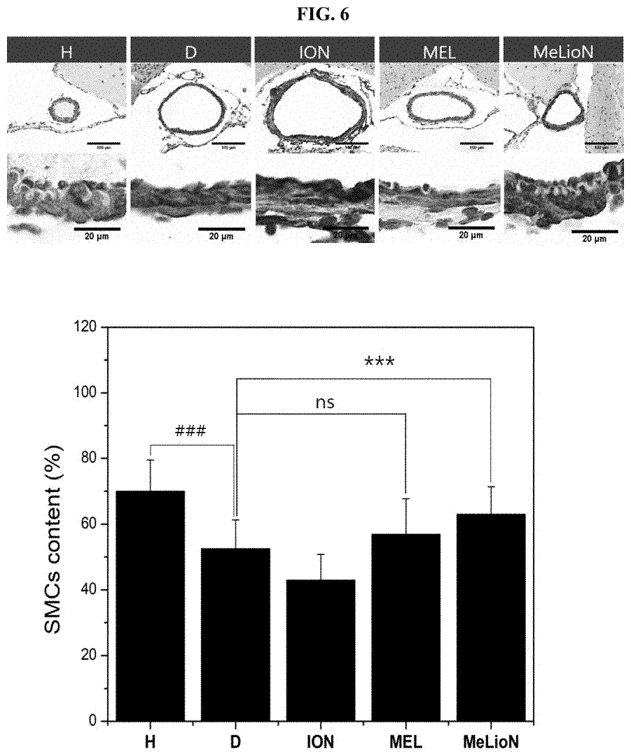

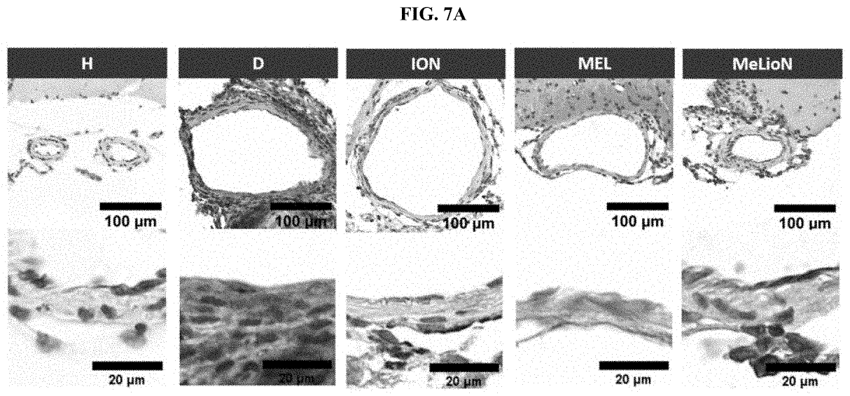

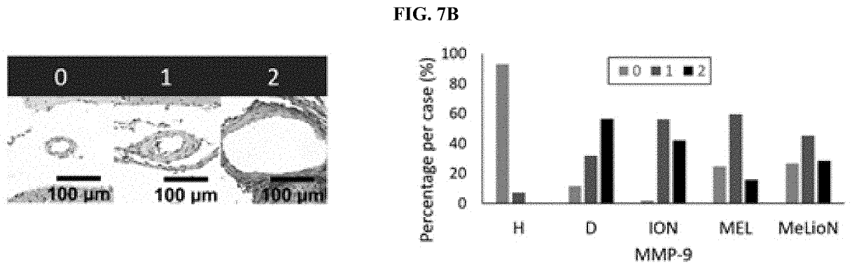

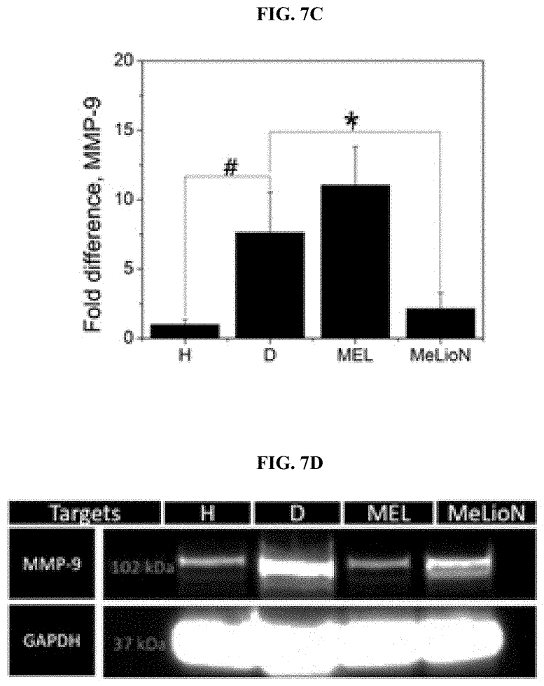

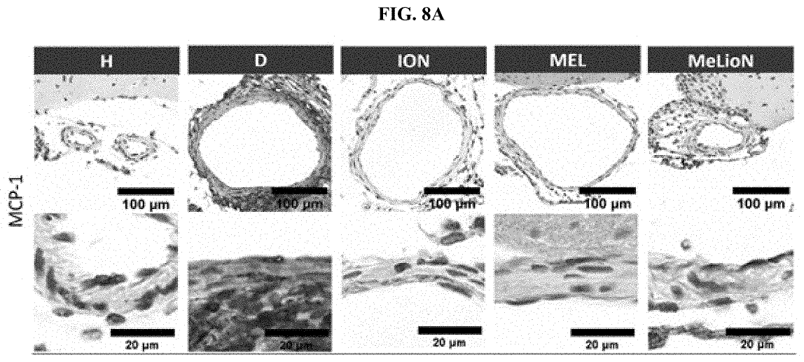

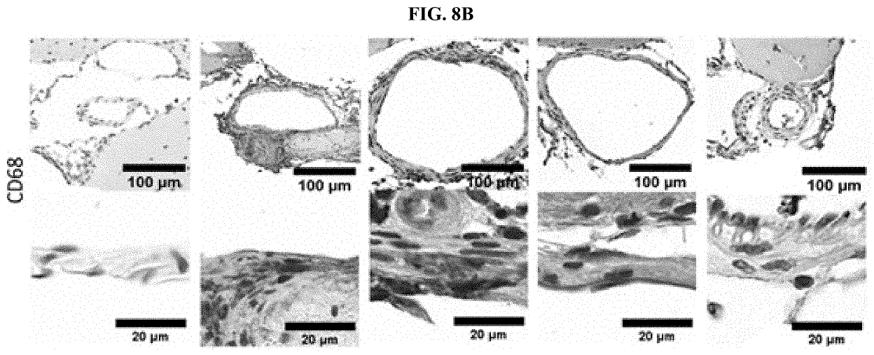

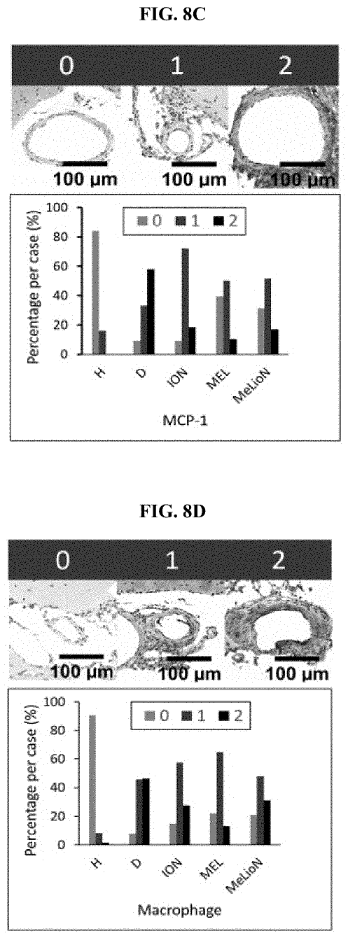

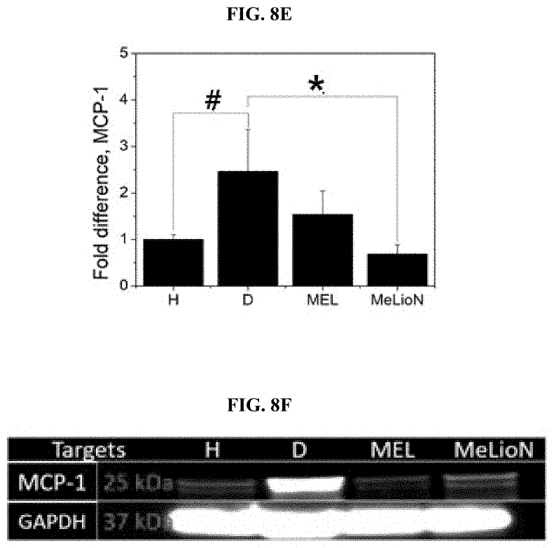

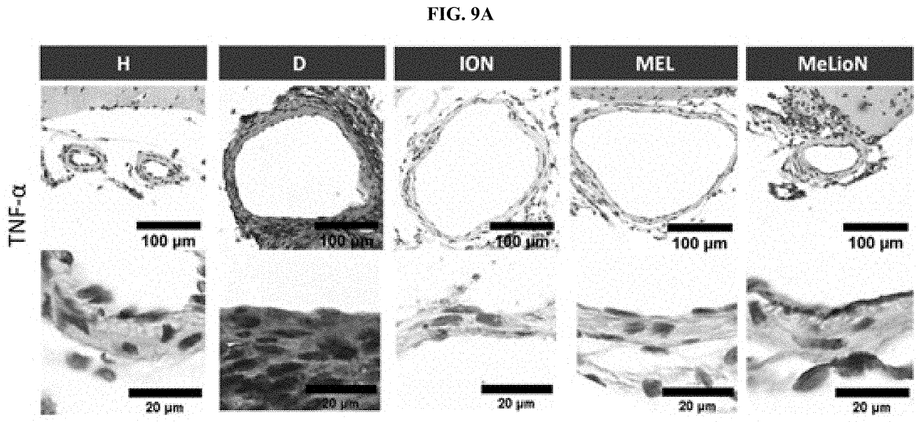

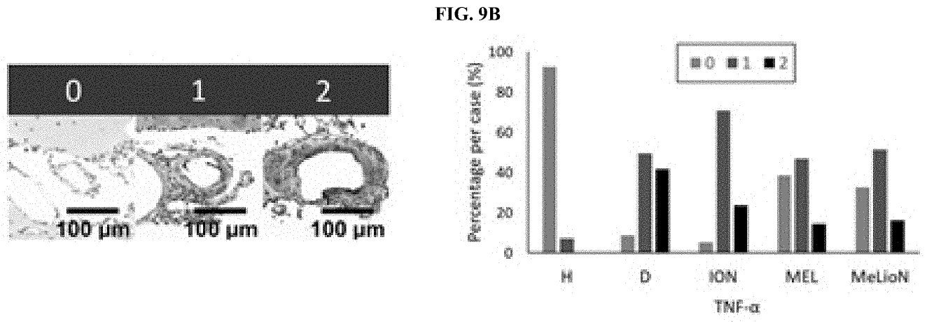

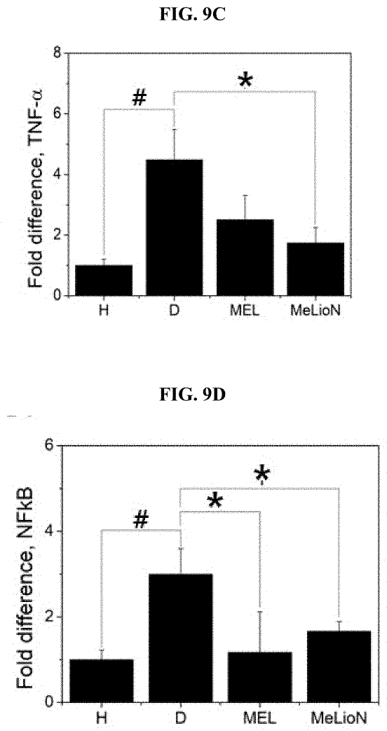

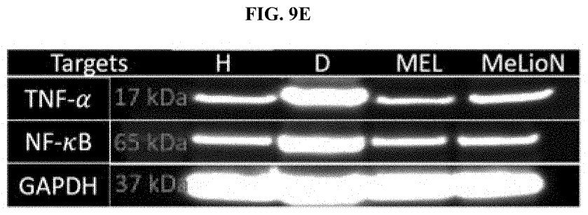

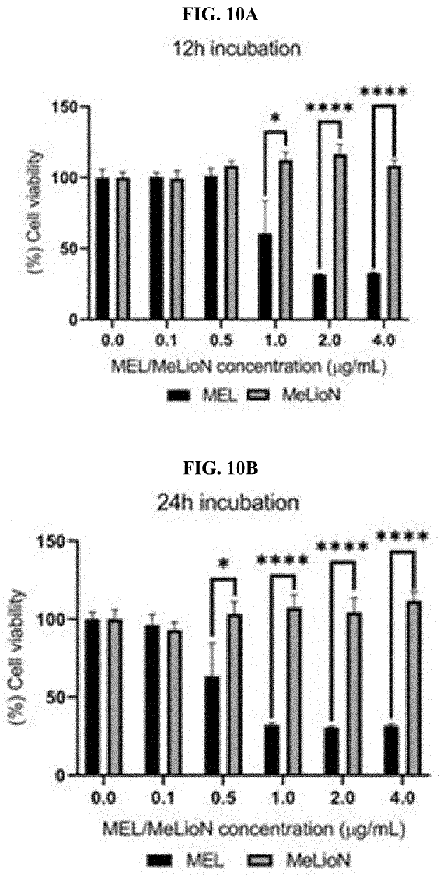

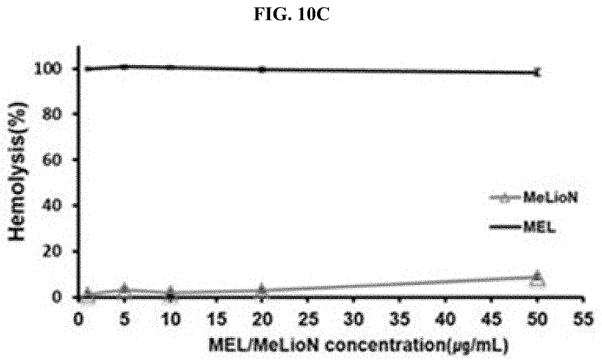

As the result are illustrated in FIGS. 3A to 3C, it was found that the diameter of the cerebral artery of the diseased group induced to have a cerebral aneurysm (D) is significantly larger than the healthy group (H), while the thickness of the cerebral artery of the diseased group induced to have a cerebral aneurysm (D) is significantly smaller than the healthy group (H). On the other hand, in the group administered with magnetic nanoparticle loaded with melittin (MeLioN), the diameter of the cerebral artery is significantly smaller and the thickness of the cerebral artery is significantly greater compared to the diseased group induced to have a cerebral aneurysm (D).

### (2) Verhoff Van Gieson Staining

Elastin content was analyzed by Verhoff Van Gieson staining technique.
As the result is illustrated in FIG. 4, it was found that the elastin content is 37.6±8.8% in the healthy group (H) while it is 5.9±3.7% in the diseased group induced to have a cerebral aneurysm (D), thus showing a statistically significant decrease (p<0.001). Furthermore, compared to the diseased group induced to have a cerebral aneurysm (D), the elastin content was 8.9±3.1% and 19.7±7.8% in the group administered only with iron oxide nanoparticle (ION) and the group administered only with melittin (MEL), respectively, thus showing no statistically significant difference. However, the elastin content was 34.9±6.2% in the group administered with magnetic iron oxide nanoparticle loaded with melittin (MeLioN), thus showing a statistically significant difference when compared to the diseased group induced to have a cerebral aneurysm (D).

### (3) Trichrome Staining

To determine the abnormal collagen content in the cerebral artery wall by Trichrome staining technique, a histopathological slide of the brain and cerebral artery located at the bottom of the brain were examined, and processed with Case Viewer software.

As the result is illustrated in FIG. 5, among the 5 groups, the collagen content was the lowest in the normal group, i.e., 8.7±3.8%. On the other hand, the group induced to have cerebral aneurysm showed the collagen content of 32.0±5.5%, thus showing an increase in statistically significant sense (p<0.001).

Furthermore, compared to the diseased group induced to have a cerebral aneurysm (D), the collagen content in the group administered only with iron oxide nanoparticle (ION) and the group administered only with melittin (MEL) showed no statistical difference. However, the collagen content was 12.6±5.1% in the group administered with magnetic iron oxide nanoparticle loaded with melittin (MeLioN) of the present invention, thus showing a statistically significant decrease compared to the diseased group induced to have a cerebral aneurysm (D).

### (4) Analysis of Fractional Ratio of Smooth Muscle Cell

The fractional ratio of smooth muscle cells was measured as follows: 70.0±9.5% for the healthy group (H), 52.5±8.8% for the diseased group induced to have a cerebral aneurysm (D), 56.9±10.8% for the group administered only with melittin (MEL), 42.9±7.9% for the group treated with iron oxide nanoparticle (ION), and 63.0±8.4% for MeLioN. It was thus found that, upon the administration, the melittin and magnetic iron oxide nanoparticle loaded with melittin of the present invention exhibit the effect of increasing the content of smooth muscles.

### Example 4. Suppressing Effect on the MMP-9 Expression in the Arterial Wall According to the Administration of Melittin (MEL) or Magnetic Iron Oxide Nanoparticle Loaded with Melittin (MeLioN) of the Present Invention

By using the brain tissues harvested in above Example 2, a change in the expression level of MMP-9 in the cerebral arterial wall was determined by immunohistochemistry (IHC) staining. The result was classified into level 0 to level 2 and subjected to statistical analysis, while it was measured by real-time PCR and Western blot.

As the result is illustrated in FIGS. 7A to 7D, the MMP-9 expression in the cerebral arterial wall was suppressed by the administration of MEL or MeLioN. The healthy group (H) showed level 0 (i.e., no distribution of MMP-9 expression was shown) while the distribution of the MMP-9 expression was higher in the diseased group induced to have a cerebral aneurysm (D). It was found that the MEL administration group and MeLioN administration group of the present invention have less level 2 (i.e., distribution of MMP-9 expression is shown in more than half of the arterial wall circumference) or less level 1 (i.e., distribution of MMP-9 expression is shown in less than half of the arterial wall circumference) compared to the diseased group induced to have a cerebral aneurysm (D).

In addition to the above, it was also found according to the real-time PCR that the expression level of MMP-9 gene is lower in the MeLioN-treated group than the diseased group induced to have a cerebral aneurysm (D), and, according to the result of Western blot analysis, the expression level of MMP-9 protein is lower in both the MEL-treated and MeLioN-treated group compared to the diseased group induced to have a cerebral aneurysm (D).

### Example 5. Suppressing Effect on the MCP-1 and CD68 Expression as Macrophage Marker in the Arterial Wall According to the Administration of Melittin (MEL) or Magnetic Iron Oxide Nanoparticle Loaded with Melittin (MeLioN) of the Present Invention

By using the brain tissues harvested in above Example 2, a change in the expression level of MCP-1 and CD68 in the cerebral arterial wall was determined by immunohistochemistry (IHC) staining. The result was classified into level 0 to level 2 and subjected to statistical analysis, while it was measured by real-time PCR and Western blot.

As the result is illustrated in FIGS. 8A to 8F, the expression level of MCP-1 and CD68 in the cerebral arterial wall was suppressed by the administration of MEL or MeLioN. The healthy group (H) mainly showed level 0 (i.e., no distribution of MCP-1 and CD68 expression was shown) while the distribution of the cells expressing MCP-1 and CD68 was higher in the group induced to have a cerebral aneurysm (D). It was found that the MEL-treated group and MeLioN-treated group of the present invention have less level 2 (i.e., expression distribution of MCP-1 and CD68 is shown in more than half of the artery wall circumference) or less level 1 (i.e., expression distribution of MCP-1 and CD68 is shown in less than half of the artery wall circumference) compared to the diseased group induced to have a cerebral aneurysm (D).

In addition to the above, it was also found according to the real-time PCR that the expression level of MCP-1 gene is lower in the MeLioN-treated group than the diseased group induced to have a cerebral aneurysm (D), and, according to the result of Western blot analysis, the expression level of MCP-1 protein is lower in both the MEL-treated group and MeLioN-treated group compared to the diseased group induced to have a cerebral aneurysm (D).

### Example 6. Determination of Suppressed Expression Level of TNF-α and NFκB in Mouse Brain Arterial Wall According to the Administration of Melittin (MEL) or Magnetic Iron Oxide Nanoparticle Loaded with Melittin (MeLioN) of the Present Invention

By using the brain tissues harvested in above Example 2, a change in the expression level of TNF-α in the cerebral arterial wall was determined by immunohistochemistry (IHC) staining. The result was classified into level 0 to level 2 and subjected to statistical analysis, while it was measured by real-time PCR and Western blot assays.

As the result is illustrated in FIGS. 9A to 9E, the expression level of TNF-α in cerebral arterial wall was suppressed by the administration of MEL or MeLioN. The healthy group (H) mainly showed level 0 (i.e., no distribution of TNF-α and NFκB expression was shown) while the distribution of the cell infiltrations expressing TNF-α and NFκB was higher in the group induced to have a cerebral aneurysm (D). It was found that the MEL administration group and MeLioN administration group of the present invention have less level 2 (i.e., expression distribution of TNF-α and NFκB is shown in more than half of the artery wall circumference) or less level 1 (i.e., expression distribution of TNF-α and NFκB is shown in less than half of the artery wall circumference) compared to the group induced to have a cerebral aneurysm (D).

In addition to the above, it was also found according to the real-time PCR that the expression level of TNF-α and NFκB genes is lower in the MEL administration group or MeLioN administration group than the group induced to have cerebral aneurysm, and, according to the result of Western blot analysis, the expression level of TNF-α and NFκB proteins is lower in both the MEL administration group and MeLioN administration group compared to the group induced to have cerebral aneurysm.

For the real-time PCR performed in above Examples 4 to 6, the primers described in the following Table 1 were used.

TABLE 1
Sequence of primers for real-time PCR

| | | SEQ |
|---|---|---|
| | | ID |
| Gene | Sequence (5′->3′) | NO: |
| TNF-α | F: CATCCGTTCTCTACCCAGCC | 2 |
| | R: AATTCTGAGCCCGGAGTTGG | 3 |
| NF-κB | F: CCTTGAAGGGATTTCCCTCC | 4 |
| | R: GGAGGGAAATCCCTTCAAGG | 5 |
| MCP-1 | F: TGATCCCAATGAGTAGGCTGGAG | 6 |
| | R: ATGTCTGGACCCATTCCTTCTTG | 7 |
| MMP-9 | F: GCCACTACTGTGCCTTTGAGTC | 8 |
| | R: CCCTCAGAGAATCGCCAGTACT | 9 |
| GAPDH | F: AGTGCCAGCCTCGTCTCATA | 10 |
| | R: GGTAACCAGGCGTCCGATAC | 11 |

### Example 7. Determination of Cytotoxicity of MEL and MeLioN

To determine any cytotoxicity exhibited by MEL and MeLioN in RAW263.7 cells, MTT assay was carried out. As the result is illustrated in FIGS. 10A to 10C, up to the concentration of 4 μg/ml, the cell viability from MeLioN was found to be similar to the group without any treatment. On the other hand, the group treated only with melittin (MEL) showed lower cell viability which is caused by cytotoxicity at the concentration of 0.5 μg/ml or higher. As such, it was determined that the treatment of melittin is preferably carried out at a concentration of less than 0.5 μg/ml.

### Statistical Analysis

Statistical analysis was performed by using Medcalc software (version 18.2.1). As for the statistical processing, to determine whether or not standard difference is present among each test group, Student's t-test and analysis of variance were carried out for comparing two groups. Diversity analysis was carried out based on Duncan's test. When p value is less than 0.05 after the statistical processing, it was found that there is a statistical significance. All data were expressed in mean±deviation, and Student's t-test was employed for mean comparison.

A sequence listing electronically submitted with the present application on Oct. 28, 2021 as an ASCII text file named 20211028_Q63721GR15_TU_SEQ, created on Sep. 27, 2021 and having a size of 3,000 bytes, is incorporated herein by reference in its entirety.

What is claimed is:

1. A method for treating a cerebrovascular disease, the method comprising:
administering a composition comprising at least one of melittin and magnetic iron oxide nanoparticle loaded with melittin as an effective component to a subject in need thereof.

2. The method of claim 1, wherein the cerebrovascular disease is anyone selected from the group consisting of cerebral aneurysm, intracranial hemorrhage and cerebral infarction.

3. The method of claim 2, wherein the cerebrovascular disease is the intracranial hemorrhage.

4. The method of claim 3, wherein the intracranial hemorrhage is subarachnoid hemorrhage, parenchymal hemorrhage, or extra-axial hemorrhage.

5. The method of claim 1, wherein the melittin consists of the amino acid sequence of SEQ ID NO: 1.

6. The method of claim 1, wherein the composition comprises the magnetic iron oxide nanoparticle loaded with the melittin.

7. The method of claim 6, wherein the magnetic iron oxide nanoparticle loaded with the melittin is produced by a method:
adding ferrous chloride and ferric chloride, admixed at 1:1 to 1:3 mole ratio, to a solution of L-arginine, and slowly adding over 1 hour a solution of ammonium hydroxide under stirring to produce a precipitated paramagnetic iron oxide nanoparticle; and
having melittin linked to a surface of the paramagnetic iron oxide nanoparticle which has been produced in the above step (1).

8. The method of claim 1, wherein the melittin or magnetic iron oxide nanoparticle loaded with melittin suppresses the expression of at least one gene or protein selected from MMP-9, MCP-1, CD68, TNF-α, and NFκB which mediate an inflammatory response.

9. The method of claim 1, wherein the composition is a pharmaceutical composition further comprising a pharmaceutically acceptable carrier, vehicle, or diluent in addition to the effective component.

10. The method of claim 1, wherein the composition is included in a functional health food.

11. The method of claim 10, wherein the composition is prepared in any one formulation selected from the group consisting of a powder, a granule, a pill, a tablet, a capsule, a candy, a syrup and a drink.
